(19) United States
(12) Patent Application Publication
(10) Pub. No.: US 2025/0107765 A1
(43) Pub. Date: Apr. 3, 2025

(54) RADIATION IMAGE PROCESSING DEVICE, RADIATION IMAGE PROCESSING METHOD, AND RADIATION IMAGE PROCESSING PROGRAM

(71) Applicant: FUJIFILM CORPORATION, Tokyo (JP)

(72) Inventor: Shin HAMAUZU, Kanagawa (JP)

(21) Appl. No.: 18/887,041

(22) Filed: Sep. 17, 2024

(30) Foreign Application Priority Data
Sep. 28, 2023 (JP) 2023-167763

(51) Int. Cl. A61B 6/00

(52) U.S. Cl. CPC A61B 6/486; A61B 6/4441

(57) ABSTRACT

A processor acquires a fluoroscopic image captured by a radiation fluoroscopy device having a C-arm, detects a plurality of feature points of a structure included in the fluoroscopic image, derives a rotation angle of the fluoroscopic image from a reference based on the plurality of detected feature points, and rotates and displays the fluoroscopic image based on the derived rotation angle.

### CROSS REFERENCE TO RELATED APPLICATIONS

The present application claims priority from Japanese Patent Application No. 2023-167763, filed on Sep. 28, 2023, the entire disclosure of which is incorporated herein by reference.

### BACKGROUND

### Technical Field

The present disclosure relates to a radiation image processing device, a radiation image processing method, and a radiation image processing program.

### Related Art

In surgical operations and catheter treatment, it is necessary to ascertain a positional relationship between surgical instruments and human body structures such as bones and blood vessels. However, in the related art, ascertaining the positional relationship between surgical instruments and human body structures often relied on the doctor's experience and intuition, resulting in problems such as incorrect insertion of surgical instruments and excessive operation times. Therefore, during operation, a patient is imaged by a radiation fluoroscopy device, and the positional relationship between the surgical instrument and the human body structure is ascertained using a radiation fluoroscopic image displayed on a display by the imaging. The radiation fluoroscopy device includes a C-arm to which a radiation source is attached at one end and to which a radiation detector is attached at the other end. In addition, the radiation fluoroscopy device is movable by rollers or the like. Then, by rotating the C-arm or moving the device itself, it is possible to image various parts of the patient in various directions.

Meanwhile, in a case in which operation is performed, a plurality of doctors, including an operating surgeon and an assistant, often work together on either side of an operating table, and fluoroscopic images need to be easiest for the operating surgeon to see. For this reason, it is preferable to dispose the C-arm of the radiation fluoroscopy device to image the patient, which is a subject, from directly beside the operating table. In a case in which the C-arm is disposed in this way, the operating surgeon can perform the work on the side opposite to the side where the C-arm is disposed on the operating table. However, the assistant has to work from the side of the C-arm, which reduces the workability of the assistant. Therefore, for example, it is considered to dispose the C-arm such that the subject is imaged from a position that does not interfere with either the operating surgeon or the assistant, such as obliquely to the side of the operating table.

However, in a case in which the C-arm is disposed in this way, the body axis of the subject included in the fluoroscopic image is inclined obliquely, which reduces the visibility of the image. For this reason, various methods have been proposed for rotating an image in which the body axis is inclined obliquely. For example, JP2009-201872A proposes a method for rotating an image in a mobile radiation imaging apparatus by estimating a direction in which the vertebrae are arranged based on edges of the vertebrae contained in a radiation image. In addition, JP2017-159192A proposes a method for a radiation fluoroscopy device to acquire an inclination angle of a landmark on a fluoroscopic image of the aorta or the like, and to rotate the fluoroscopic image based on the acquired inclination angle so that the landmark is vertical or horizontal.

However, the mobile radiation imaging apparatus disclosed in JP2009-201872A is not intended to capture images of a subject during operation or treatment. Furthermore, the method disclosed in JP2017-159192A requires detection of the aorta itself as a landmark, and therefore requires a long period of time for detection processing.

### SUMMARY OF THE INVENTION

The present disclosure has been made in consideration of the above circumstances, and an object of the present disclosure is to easily achieve both workability in surgical operation and the like and visibility of acquired fluoroscopic images.

According to an aspect of the present disclosure, there is provided a radiation image processing device comprising at least one processor, in which the processor is configured to: acquire a fluoroscopic image captured by a radiation fluoroscopy device having a C-arm;

detect a plurality of feature points of a structure included in the fluoroscopic image;

derive a rotation angle of the fluoroscopic image from a reference based on the plurality of detected feature points; and

rotate and display the fluoroscopic image based on the derived rotation angle.

In the radiation image processing device according to the aspect of the present disclosure, in a case in which the radiation fluoroscopy device continuously acquires the fluoroscopic images, the processor may be configured to: detect the feature points and derive the rotation angle each time the fluoroscopic image is acquired at a predetermined interval; and

in a case in which a change in a newly derived rotation angle from a previously derived rotation angle is less than a reference angle, rotate and display the fluoroscopic image by the same rotation angle as a rotation angle in a case in which the previously acquired fluoroscopic image was displayed.

In addition, in the radiation image processing device according to the aspect of the present disclosure, the processor may be configured to, in a case in which the newly derived rotation angle has changed by the reference angle or more from the previously derived rotation angle, rotate and display the fluoroscopic image by the newly derived rotation angle.

In addition, in the radiation image processing device according to the aspect of the present disclosure, the processor may be configured to: detect whether or not the C-arm has moved or the radiation fluoroscopy device has moved; and

in a case in which the movement of the C-arm or the movement of the radiation fluoroscopy device is detected, rotate and display the fluoroscopic image by the newly derived rotation angle.

In addition, in the radiation image processing device according to the aspect of the present disclosure, the structure may be a plurality of vertebrae,

the feature points may be centroids of each of the plurality of vertebrae, and

the processor may be configured to: derive an approximate straight line connecting the centroids of the plurality of vertebrae; and

rotate the fluoroscopic image such that the approximate straight line has an orientation of the reference.

According to another aspect of the present disclosure, there is provided a radiation image processing method executed by a computer, the radiation image processing method comprising: acquiring a fluoroscopic image captured by a radiation fluoroscopy device having a C-arm;

detecting a plurality of feature points of a structure included in the fluoroscopic image;

deriving a rotation angle of the fluoroscopic image from a reference based on the plurality of detected feature points; and

rotating and displaying the fluoroscopic image based on the derived rotation angle.

According to still another aspect of the present disclosure, there is provided a radiation image processing program causing a computer to execute: a step of acquiring a fluoroscopic image captured by a radiation fluoroscopy device having a C-arm;

a step of detecting a plurality of feature points of a structure included in the fluoroscopic image;

a step of deriving a rotation angle of the fluoroscopic image from a reference based on the plurality of detected feature points; and

a step of rotating and displaying the fluoroscopic image based on the derived rotation angle.

According to the aspects of the present disclosure, it is possible to easily achieve both workability in surgical operation and the like and visibility of acquired fluoroscopic images.

### BRIEF DESCRIPTION OF THE DRAWINGS

FIG. 1 is a schematic diagram of a fluoroscopy system to which a radiation image processing device according to a first embodiment of the present disclosure is applied.

FIG. 2 is a diagram showing a hardware configuration of the radiation image processing device according to the first embodiment.

FIG. 3 is a diagram showing a functional configuration of the radiation image processing device according to the first embodiment.

FIG. 4A and 4B are diagrams showing a state in which a C-arm is inserted from directly beside an operating table and a fluoroscopic image acquired in that state.

FIGS. 5A and 5B are diagrams showing a state in which a C-arm is inserted into an operating table in an oblique direction and a fluoroscopic image acquired in that state.

FIG. 6 is a diagram for describing a centroid.

FIG. 7 is a diagram showing a result of detection of feature points.

FIG. 8 is a diagram for describing calculation of an approximate straight line.

FIG. 9 is a diagram showing a fluoroscopic image displayed after being rotated.

FIG. 10 is a flowchart showing a process performed in the first embodiment.

FIG. 11 is a schematic diagram of a fluoroscopy system to which a radiation image processing device according to a second embodiment of the present disclosure is applied.

FIG. 12 is a flowchart showing a process performed in the second embodiment.

### DETAILED DESCRIPTION

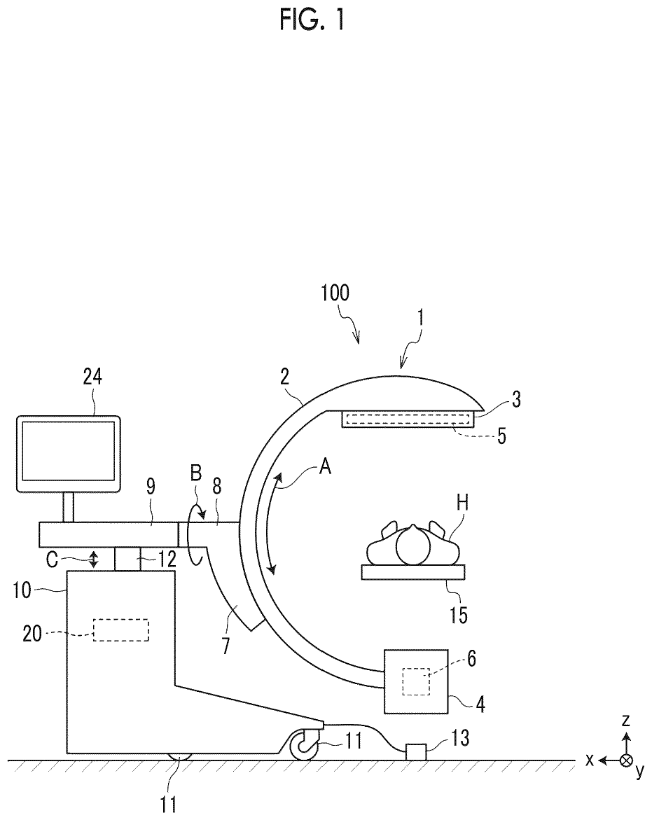

Hereinafter, embodiments of the present disclosure will be described with reference to the drawings. FIG. 1 is a schematic diagram showing a configuration of a fluoroscopy system including a radiation image processing device according to a first embodiment of the present disclosure. As shown in FIG. 1, a fluoroscopy system 100 according to the first embodiment comprises a fluoroscopy apparatus 1.

As shown in FIG. 1, the fluoroscopy apparatus 1 according to the present embodiment comprises a C-arm 2. A detection unit 3 is attached to one end part of the C-arm 2, and a radiation emitting unit 4 is attached to the other end part of the C-arm 2 to face the detection unit 3.

The configuration of the fluoroscopy apparatus 1 will be described below in detail. A radiation detector 5, such as a flat panel detector, is provided in the detection unit 3. In addition, for example, a circuit board including a charge amplifier that converts a charge signal read out from the radiation detector 5 into a voltage signal, a sampling two correlation pile circuit that samples the voltage signal output from the charge amplifier, and an analog-digital (AD) conversion unit that converts the voltage signal into a digital signal is also provided in the detection unit 3. Further, in the present embodiment, the radiation detector 5 is used. On the other hand, the present embodiment is not limited to the radiation detector 5 as long as radiation can be detected and the radiation can be converted into an image. For example, a detection device such as an image intensifier can be used.

The radiation detector 5 can repeatedly perform recording and reading out of a radiation image, may be a so-called direct-type radiation detector that directly converts radiation such as X-rays into charges, or may be a so-called indirect-type radiation detector that converts radiation into visible light once and converts the visible light into a charge signal. As a method for reading out a radiation image signal, it is desirable to use the following method: a so-called thin film transistor (TFT) readout method which reads out a radiation image signal by turning on and off a TFT switch; or a so-called optical readout method which reads out a radiation image signal by irradiating a target with readout light. On the other hand, the readout method is not limited thereto, and other methods may be used.

A radiation source 6 is accommodated in the radiation emitting unit 4, and the radiation source 6 emits radiation toward the detection unit 3. The radiation source 6 emits X-rays as radiation, and a timing at which the radiation source 6 emits radiation and a timing at which the radiation detector 5 detects the radiation are controlled by an imaging controller, which will be described later. In addition, the radiation generation conditions in the radiation source 6, that is, the selection of the material of the target and the filter, the tube voltage, the irradiation time, and the like are also controlled by the imaging controller.

The C-arm 2 according to the present embodiment is held by a C-arm holding part 7 to be movable in the direction of an arrow A shown in FIG. 1, and integrally changeable in angle with respect to the detection unit 3 and the radiation emitting unit 4 in the z direction (vertical direction) shown in FIG. 1. In addition, the C-arm holding part 7 includes a shaft part 8, and the shaft part 8 rotatably connects the C-arm 2 to a bearing 9. Thereby, the C-arm 2 is configured to be rotatable in the direction of an arrow B shown in FIG. 1 with the shaft part 8 as a rotation axis.

In addition, as shown in FIG. 1, the fluoroscopy apparatus 1 comprises a body part 10. A plurality of wheels 11 are attached to a bottom portion of the body part 10, and thus, the fluoroscopy apparatus 1 can be moved. A support shaft 12 that is expanded and contracted in a z-axis direction of FIG. 1 is provided on an upper portion side of a housing of the body part 10 in FIG. 1. The bearing 9 is held on the upper portion of the support shaft 12 to be movable in the direction of an arrow C. Thus, the C-arm 2 can be moved in an up-down direction with respect to an operating table 15.

In addition, a foot switch 13 for turning on and off the emission of radiation from the radiation source 6 of the radiation emitting unit 4 is connected to the body part 10. A doctor during operation steps on the foot switch 13 to turn it on, which causes the radiation source 6 to emit radiation in a pulsed manner at a predetermined interval. In a case in which the doctor removes his/her foot from the foot switch 13, it is turned off, and thus the emission of the radiation from the radiation source 6 is stopped. The predetermined interval may be a time interval.

The fluoroscopy apparatus 1 has the above-described configuration, and thus, irradiates the subject H from below the subject H who is lying on the operating table 15 with radiation, detects the pulsed radiation transmitted through the subject H with the radiation detector 5 of the detection unit 3, and continuously acquires fluoroscopic images of the subject H from the front in accordance with the timing of the emission of the radiation.

Here, the C-arm 2 is movable in the direction of the arrow A, the direction of the arrow B, and the direction of the arrow C, and the fluoroscopy apparatus 1 is movable by the wheels 11. Therefore, the fluoroscopy apparatus 1 can image a desired part of the subject H who is lying on the operating table 15 in a desired direction while adjusting their own positions and the position of the C-arm 2.

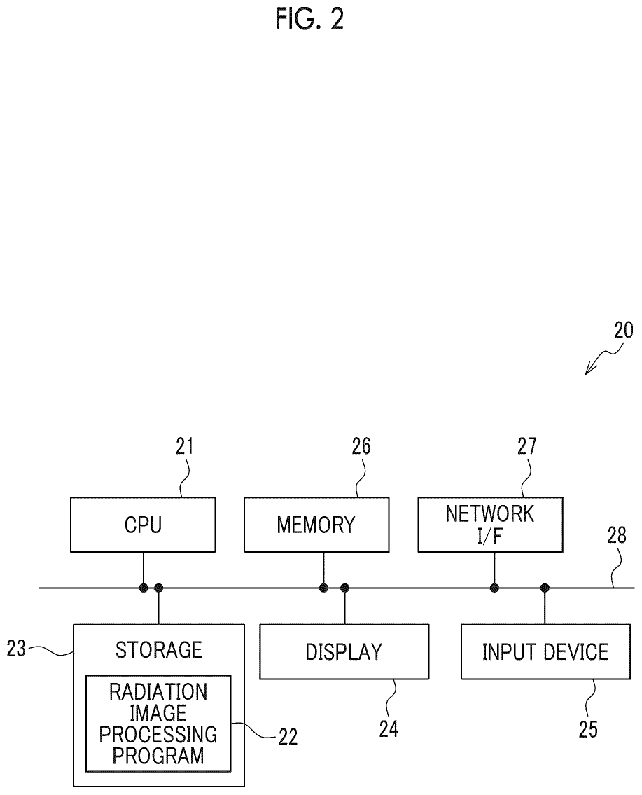

A radiation image processing device 20 according to the first embodiment is built in the body part 10. FIG. 2 is a diagram showing a hardware configuration of the radiation image processing device according to the first embodiment. As shown in FIG. 2, the radiation image processing device 20 is a computer, such as a workstation, a server computer, and a personal computer, and comprises a central processing unit (CPU) 21, a non-volatile storage 23, and a memory 26 as a temporary storage area. In addition, the radiation image processing device 20 comprises a display 24, such as a liquid crystal display, an input device 25 such as a keyboard and a mouse, and a wired or wireless interface (I/F) 27 that is connected to the detection unit 3, the radiation emitting unit 4, and the foot switch 13, and is used to exchange information with external devices. The CPU 21, the storage 23, the display 24, the input device 25, the memory 26, and the I/F 27 are connected to a bus 28. The CPU 21 is an example of a processor according to the present disclosure.

The storage 23 is realized by a hard disk drive (HDD), a solid-state drive (SSD), a flash memory, and the like. A radiation image processing program 22 installed in the radiation image processing device 20 is stored in the storage 23 serving as a storage medium. The CPU 21 reads out the radiation image processing program 22 from the storage 23, loads the read program into the memory 26, and executes the loaded radiation image processing program 22.

The radiation image processing program 22 is stored in a storage device of a server computer connected to the network or in a network storage in a state in which it can be accessed from the outside, and is downloaded to and installed on the radiation image processing device 20 in response to a request. Alternatively, the radiation image processing program 22 is recorded on a recording medium, such as a digital versatile disc (DVD) and a compact disc read-only memory (CD-ROM), and distributed, and is installed on the radiation image processing device 20 from the recording medium.

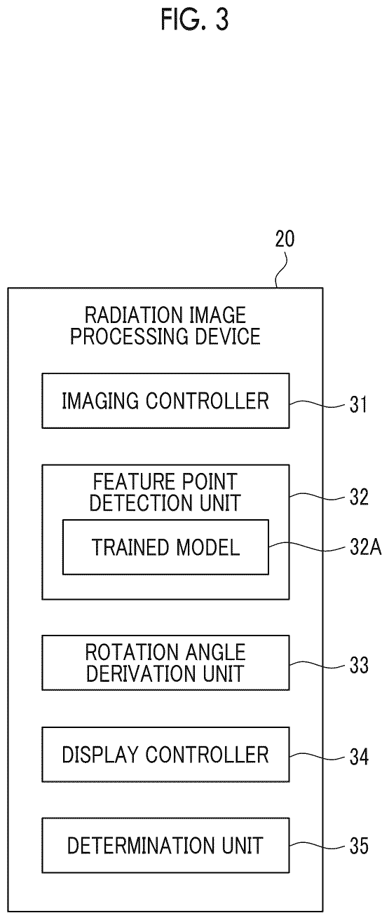

Next, a functional configuration of the radiation image processing device according to the first embodiment will be described. FIG. 3 is a diagram showing a functional configuration of the radiation image processing device according to the first embodiment. As shown in FIG. 3, the radiation image processing device 20 comprises an imaging controller 31, a feature point detection unit 32, a rotation angle derivation unit 33, a display controller 34, and a determination unit 35. Then, in a case in which the CPU 21 executes the radiation image processing program 22, the CPU 21 functions as the imaging controller 31, the feature point detection unit 32, the rotation angle derivation unit 33, the display controller 34, and the determination unit 35.

In a case in which the foot switch 13 is turned on and an on signal from the foot switch 13 is input, the imaging controller 31 causes radiation to be emitted from the radiation source 6 included in the radiation emitting unit 4 based on preset imaging conditions. Furthermore, the imaging controller 31 detects the radiation transmitted through the subject H with the radiation detector 5 of the detection unit 3 in response to the timing at which the radiation is emitted from the radiation source 6, and generates a fluoroscopic image of the subject H. The generated fluoroscopic image is displayed on the display 24.

In the present embodiment, the imaging controller 31 controls the radiation source 6 to emit the radiation in a pulsed manner at a predetermined interval while the foot switch 13 is turned on. Accordingly, the pulsed radiation is emitted from the radiation source 6, and the fluoroscopic image is generated by the radiation detector 5 at a timing corresponding to the emission of the radiation. Therefore, the fluoroscopic images are continuously displayed on the display 24 like a moving image at a frame rate corresponding to the emission interval of the pulsed radiation.

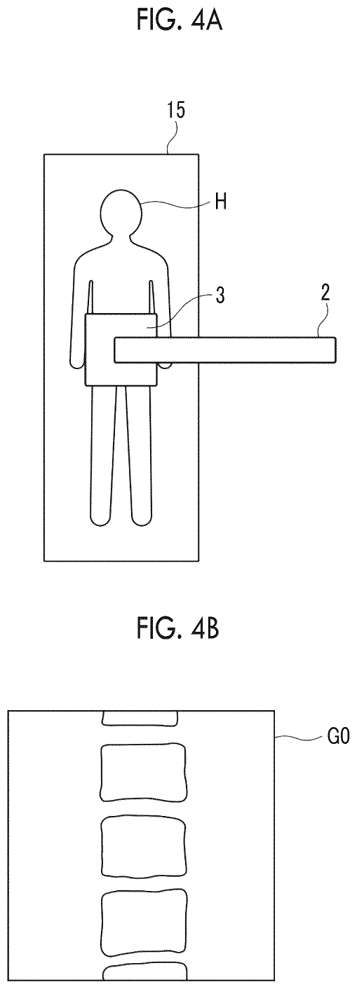

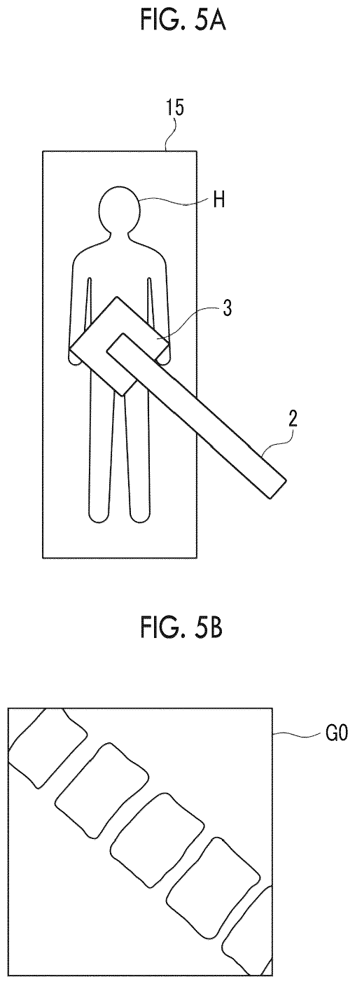

Here, in a case in which the C-arm 2 is inserted directly beside the operating table 15 to image the waist of the subject H as shown in FIG. 4A, in an acquired fluoroscopic image G0, the direction in which the vertebrae are arranged, that is, the body axis of the subject H, is perpendicular to the fluoroscopic image G0 as shown in FIG. 4B. On the other hand, in a case in which doctors (for example, an operating surgeon and an assistant) are working on both sides of the operating table 15, the C-arm 2 cannot be inserted directly beside the operating table 15. In this case, in order not to interfere with the work, the C-arm 2 is inserted into the operating table 15 in an oblique direction as shown in FIG. 5A. In a case in which the C-arm 2 is inserted into the operating table 15 in the oblique direction in this manner, in the acquired fluoroscopic image G0, the body axis of the subject H is inclined with respect to each side of the fluoroscopic image G0, as shown in FIG. 5B. The fluoroscopic image G0 is displayed on the display 24 such that each side of the fluoroscopic image G0 is parallel to each side of the display 24. For this reason, in the fluoroscopic image G0 displayed on the display 24, the body axis of the subject H is inclined. The fluoroscopic image G0 with the body axis inclined in this way has poor visibility, and there is a likelihood that the work cannot be performed accurately.

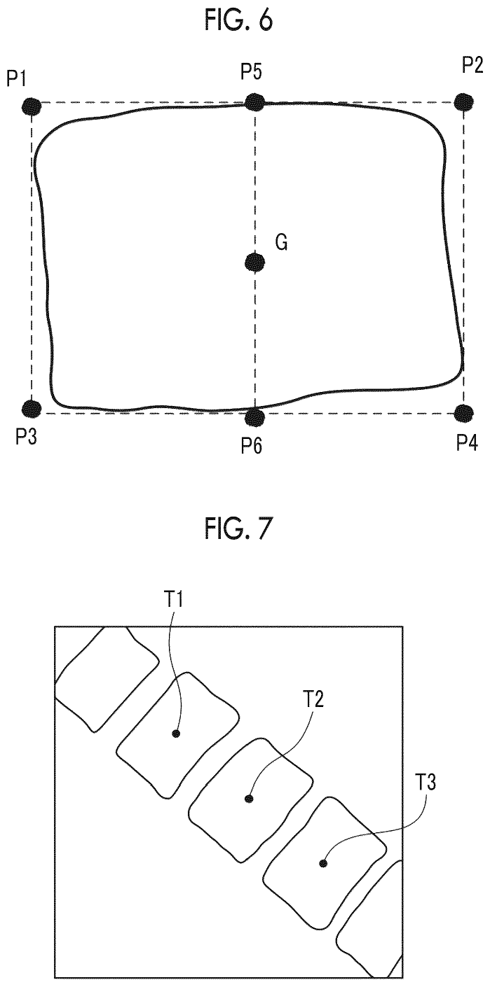

To this end, in the present embodiment, first, the feature point detection unit 32 detects a plurality of feature points of the structure included in the fluoroscopic image G0. In the present embodiment, a centroid of the vertebra of the subject H included in the fluoroscopic image G0 is detected as a feature point. FIG. 6 is a diagram for describing a centroid. As shown in FIG. 6, first, a circumscribing rectangle that circumscribes the vertebra is defined. Then, among four vertices P1 to P4 of the circumscribing rectangle, a midpoint P5 of the vertices P1 and P2 and a midpoint P6 of the vertices P3 and P4 are derived. The midpoint of the midpoints P5 and P6 is a centroid G of the vertebrae. The feature point detection unit 32 in the present embodiment has a trained model 32A in which machine learning is performed to detect the centroid of the vertebra from the fluoroscopic image G0. The trained model 32A is constructed by training a neural network using fluoroscopic images for learning including, for example, vertebrae and correct answer data in which the centroids of the vertebrae in the fluoroscopic images for learning are labeled as training data. Note that a feature point may be one pixel, or a small region consisting of a plurality of pixels may be detected as a feature point.

FIG. 7 is a diagram showing a result of detection of feature points. As shown in FIG. 7, the feature point detection unit 32 detects feature points T1 to T3 for each of the three vertebrae included in the fluoroscopic image G0.

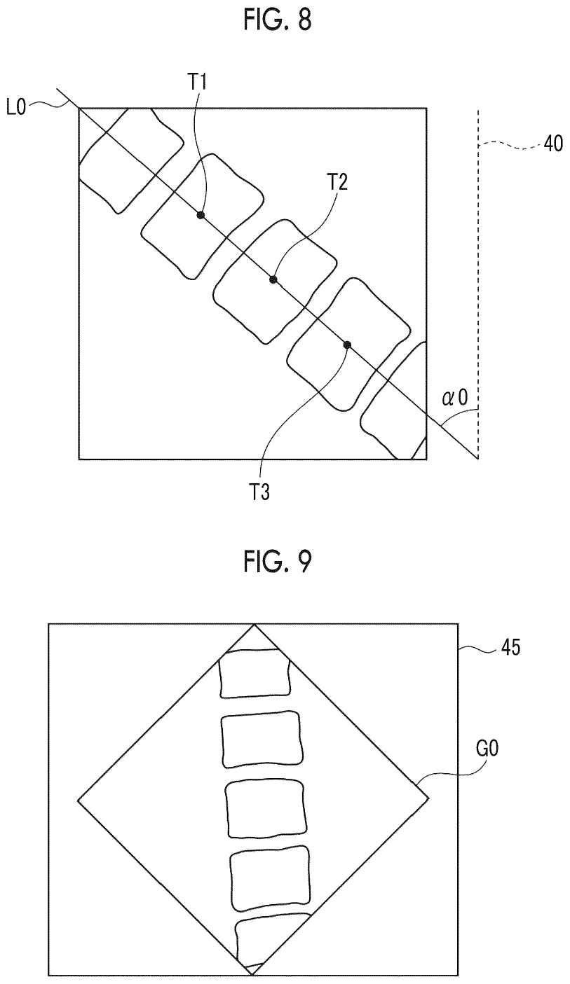

The rotation angle derivation unit 33 derives a rotation angle of the fluoroscopic image G0 from a reference based on the plurality of feature points detected by the feature point detection unit 32. To this end, the rotation angle derivation unit 33 first derives an approximate straight line passing through the plurality of feature points T1 to T3. The rotation angle derivation unit 33 derives a straight line passing near the feature points T1 to T3 as an approximate straight line using, for example, methods such as a principal component analysis and a least squares method. FIG. 8 is a diagram showing an approximate straight line L0 passing through the three feature points T1 to T3. The rotation angle derivation unit 33 derives a rotation angle α0 of the approximate straight line L0 with a vertical axis 40 of the fluoroscopic image G0 as a reference. In FIG. 8, a counterclockwise rotation angle with respect to the vertical axis 40 is defined as a positive rotation angle, but the present disclosure is not limited thereto.

Here, in the present embodiment, the feature point detection unit 32 and the rotation angle derivation unit 33 detect feature points and derive a rotation angle each time the fluoroscopic images G0 are continuously acquired. That is, the detection of the feature points and the derivation of the rotation angle are performed for all the fluoroscopic images G0 continuously acquired. Note that the detection of the feature points and the derivation of the rotation angle may be performed at a predetermined interval for the fluoroscopic images G0 continuously acquired. For example, the detection of the feature points and the derivation of the rotation angle may be performed may be performed at predetermined frame intervals, such as a 5-frame interval or a 10-frame interval.

The display controller 34 rotates the fluoroscopic image G0 based on the rotation angle α0 derived by the rotation angle derivation unit 33 and displays the fluoroscopic image G0 on the display 24. Specifically, the fluoroscopic image G0 is rotated by −α0 degrees around the center point of the fluoroscopic image G0. Accordingly, the fluoroscopic image G0 is displayed on the display 24 such that the vertebral direction in which the vertebrae included in the fluoroscopic image G0 are arranged matches the up-down direction of the display 24. FIG. 9 is a diagram showing a display screen of a fluoroscopic image. As shown in FIG. 9, on a display screen 45, the fluoroscopic image G0 is rotated by −α0 degrees, and thereby the plurality of vertebrae are displayed arranged in the up-down direction of the display 24.

In a case in which the C-arm 2 is inserted from directly beside the operating table 15 as shown in FIG. 4A, the rotation angle α0 of the fluoroscopic image G0 is approximately 0 degrees. Even in this case, in the present embodiment, the image is rotated by −α0 degrees that is approximately 0 degrees and displayed. Therefore, the acquired fluoroscopic image G0 is displayed on the display 24 as if it is not substantially rotated.

The determination unit 35 compares the rotation angle (defined as αnew) derived by the rotation angle derivation unit 33 for the latest fluoroscopic image (defined as Gnew) among the continuously acquired fluoroscopic images G0 with the rotation angle (defined as αold) derived by the rotation angle derivation unit 33 for the previously acquired fluoroscopic image (defined as Gold). The latest fluoroscopic image Gnew is a fluoroscopic image acquired by the most recent emission of radiation. The previous fluoroscopic image Gold is a fluoroscopic image acquired by emitting radiation at a timing immediately before the most recent emission of radiation. Then, the determination unit 35 determines whether or not the change in the newly derived rotation angle αnew from the previously derived rotation angle αold is less than a predetermined reference angle Th0, that is, whether or not |αnew−αold|<Th0. In a case in which |αnew−αold|<Th0, the display controller 34 is instructed to rotate and display the latest fluoroscopic image Gnew by the same rotation angle as the rotation angle in a case in which the previously acquired fluoroscopic image Gold was displayed. The reference angle Th0 can be, for example, 5 to 10 degrees, but the present disclosure is not limited thereto.

On the other hand, in a case in which the newly derived rotation angle αnew has changed from the previously derived rotation angle αold by the reference angle Th0 or more, that is, in a case in which |αnew−αold|>Th0, the determination unit 35 instructs the display controller 34 to rotate and display the latest fluoroscopic image Gnew by the newly derived rotation angle αnew.

Accordingly, even in a case in which the rotation angle αnew changes slightly, as long as |αnew−αold|<Th0 is satisfied, the latest fluoroscopic image Gnew is rotated by the rotation angle derived at the start of processing or the rotation angle when |αnew−αold| lastly becomes ≥Th0 and is displayed on the display 24.

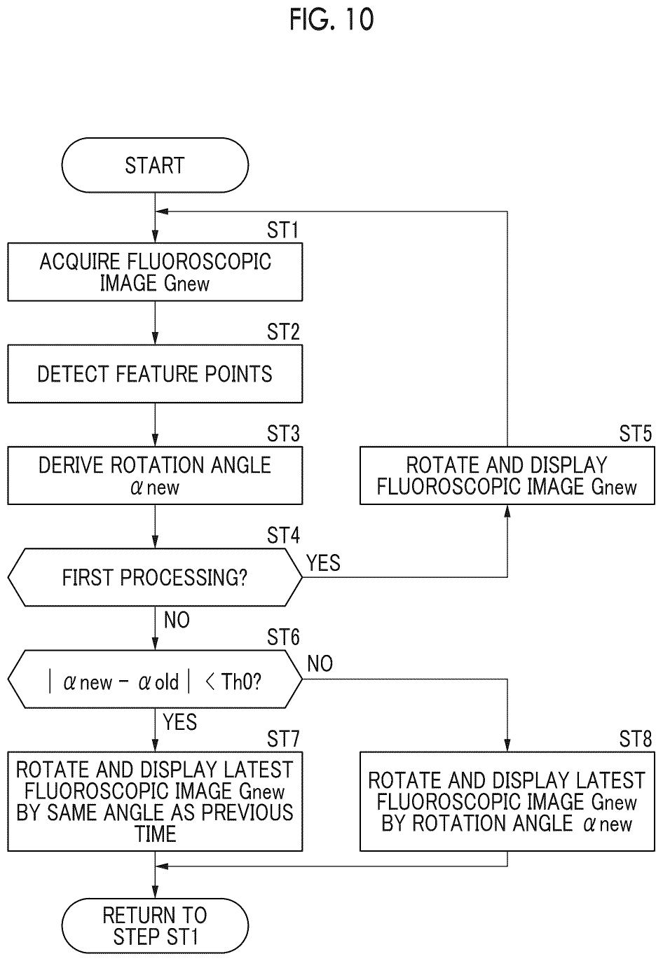

Next, a process performed in the first embodiment will be described. FIG. 10 is a flowchart showing a process performed in the first embodiment. It is assumed that the imaging controller 31 continuously acquires the fluoroscopic images G0 at a predetermined frame rate. Also, it is assumed that Gnew is used as a reference numeral for the latest fluoroscopic image, Gold is used as a reference numeral for the previous fluoroscopic image, αnew is used as a reference numeral for the latest rotation angle, and αold is used as a reference numeral for the previous rotation angle. In a case in which an instruction to start imaging is given, the latest fluoroscopic image Gnew is acquired (Step ST1). Then, the feature point detection unit 32 detects feature points from the fluoroscopic image Gnew (Step ST2), and the rotation angle derivation unit 33 derives the rotation angle αnew of the fluoroscopic image Gnew (Step ST3).

Next, the determination unit 35 determines whether or not this is the first processing (Step ST4). In a case in which a determination result in Step ST4 is “Yes”, the display controller 34 rotates the fluoroscopic image Gnew by the derived rotation angle αnew and displays the fluoroscopic image Gnew on the display 24 (Step ST5), and then the process returns to Step ST1.

In a case in which a determination result in Step ST4 is “No”, the determination unit 35 compares the rotation angle αnew derived by the rotation angle derivation unit 33 for the latest fluoroscopic image Gnew with the rotation angle αold derived by the rotation angle derivation unit 33 for the previously acquired fluoroscopic image Gold, and determines whether or not |αnew−αold|<Th0 (Step ST6).

In a case in which a determination result in Step ST6 is “Yes”, the determination unit 35 instructs the display controller 34 to rotate and display the latest fluoroscopic image Gnew by the same rotation angle as the rotation angle in a case in which the previously acquired fluoroscopic image Gold was displayed. Accordingly, the display controller 34 rotates the fluoroscopic image Gnew by the same rotation angle as the rotation angle in a case in which the previously acquired fluoroscopic image Gold was displayed, displays the fluoroscopic image Gnew on the display 24 (Step ST7), and then the process returns to Step ST1.

In a case in which a determination result in Step ST6 is “No”, the determination unit 35 instructs the display controller 34 to rotate and display the latest fluoroscopic image Gnew by the latest rotation angle αnew. Accordingly, the display controller 34 rotates the fluoroscopic image Gnew by the rotation angle αnew, displays the fluoroscopic image Gnew on the display 24 (Step ST8), and then the process returns to Step ST1.

In this way, in the present embodiment, a plurality of feature points of a structure included in the fluoroscopic image are detected, a rotation angle of the fluoroscopic image from a reference is derived based on the plurality of detected feature points, and the fluoroscopic image is rotated and displayed based on the derived rotation angle. Therefore, the fluoroscopic image can be rotated and displayed by an operation that is simpler than extracting the structure itself. Therefore, it is possible to easily achieve both workability in surgical operation and the like and visibility of acquired fluoroscopic images.

In addition, in a case in which the change in the newly derived rotation angle αold from the previously derived rotation angle is less than the reference angle Th0, the fluoroscopic image Gnew is rotated and displayed by the same rotation angle αold as the rotation angle αold in a case in which the previously acquired fluoroscopic image Gold was displayed. Therefore, it is possible to prevent the fluoroscopic images G0 from being finely rotated and displayed in accordance with a slight change in rotation angle, and as a result, it is possible to improve the visibility of the displayed continuously fluoroscopic images G0.

In addition, in a case in which the newly derived rotation angle αnew has changed from the previously derived rotation angle αold by the reference angle Th0 or more, the fluoroscopic image Gnew is rotated by the newly derived rotation angle αnew and displayed. Therefore, even in a case in which the rotation angle of the fluoroscopic image G0 is significantly changed by moving the fluoroscopy apparatus 1 or the C-arm 2, the fluoroscopic image G0 can be displayed in a desired orientation. Therefore, the visibility of the continuously displayed fluoroscopic images G0 can be improved.

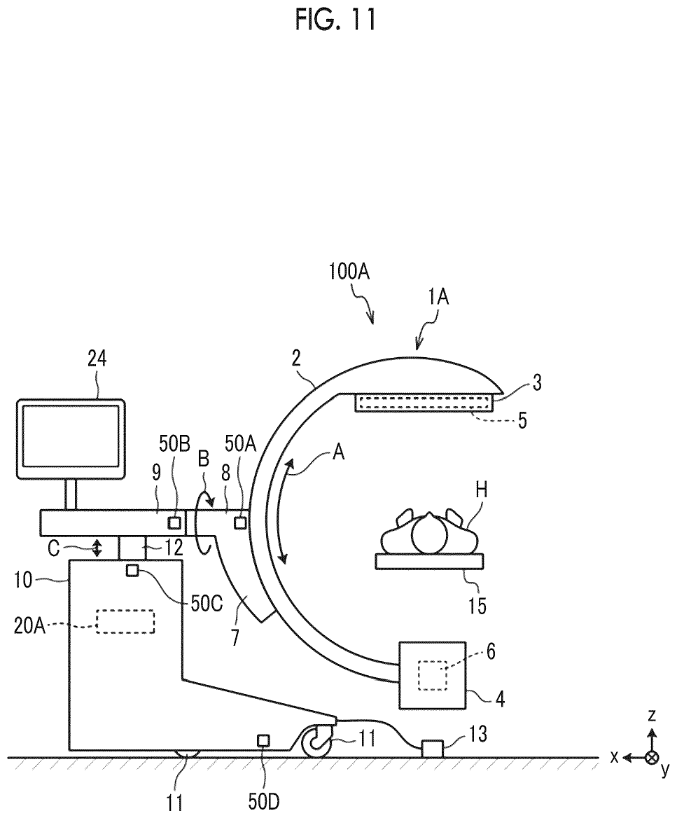

Next, a second embodiment of the present disclosure will be described. FIG. 11 is a schematic diagram showing a configuration of a fluoroscopy system including a radiation image processing device according to the second embodiment of the present disclosure. In FIG. 11, the same reference numerals are assigned to the same configurations as those in FIG. 1, and detailed description thereof will be omitted. A fluoroscopy system 100A comprising a radiation image processing device 20A according to the second embodiment is different from the first embodiment in that the fluoroscopy system 100A uses a fluoroscopy apparatus 1A comprising four sensors 50A to 50D.

The sensor 50A is provided in the C-arm holding part 7 and detects movement of the C-arm 2 in the direction of the arrow A. The sensor 50B is provided in the bearing 9 and detects the rotation of the C-arm 2 in the direction of the arrow B. The sensor 50C is provided in an upper portion of the body part 10 and detects the movement of the C-arm 2 in the direction of the arrow C. The sensor 50D is provided in a lower portion of the body part 10 and detects the movement of the fluoroscopy apparatus 1A.

In addition, since the hardware configuration and the functional configuration of the radiation image processing device 20A according to the second embodiment are the same as those of the radiation image processing device 20 according to the first embodiment, detailed description thereof will be omitted here.

In the second embodiment, the determination unit 35 detects the movement of the C-arm 2 (movement in the direction of the arrow A, rotation in the direction of the arrow B, and movement in the direction of the arrow C) with the sensors 50A to 50C, and detects the movement of the fluoroscopy apparatus 1A with the sensor 50D. In a case in which the movement of the C-arm 2 or the movement of the fluoroscopy apparatus 1A is detected, the determination unit 35 instructs the display controller 34 to rotate and display the latest fluoroscopic image Gnew by the newly derived rotation angle αnew, and the display controller 34 rotates and displays the fluoroscopic image Gnew by the rotation angle αnew. The movement of the C-arm 2 includes at least one of the movement of the C-arm 2 in the direction of the arrow A, the rotation of the C-arm 2 in the direction of the arrow B, or the movement of the C-arm 2 in the direction of the arrow C.

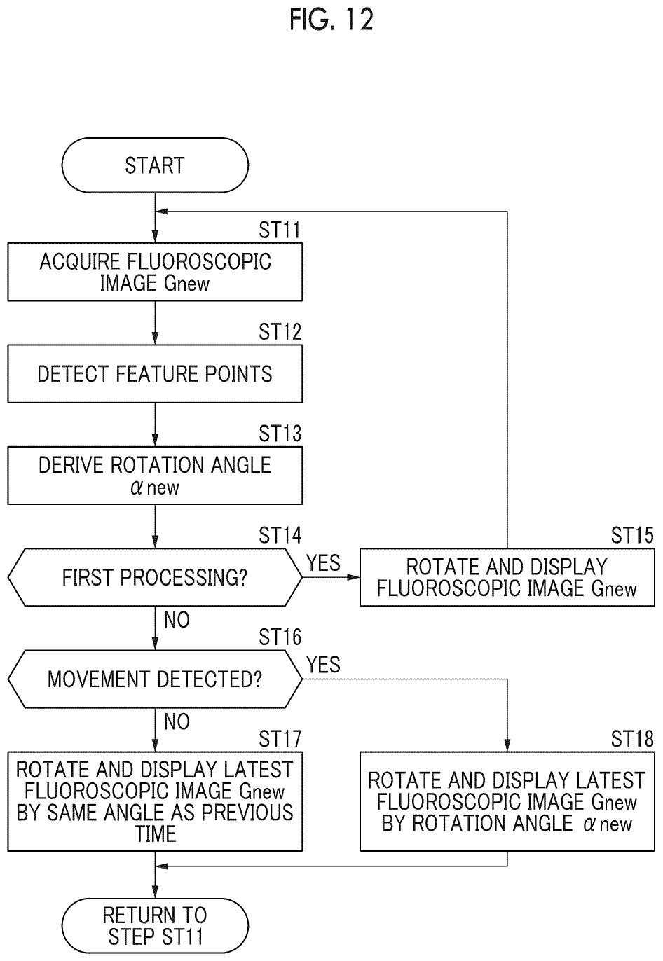

Next, a process performed in the second embodiment will be described. FIG. 12 is a flowchart showing a process performed in the second embodiment. It is assumed that the imaging controller 31 continuously acquires the fluoroscopic images G0 at a predetermined frame rate. In a case in which an instruction to start imaging is given, the latest fluoroscopic image Gnew is acquired (Step ST11). Then, the feature point detection unit 32 detects feature points from the fluoroscopic image Gnew (Step ST12), and the rotation angle derivation unit 33 derives the rotation angle αnew of the fluoroscopic image Gnew (Step ST13).

Next, the determination unit 35 determines whether or not this is the first processing (Step ST14). In a case in which a determination result in Step ST14 is “Yes”, the display controller 34 rotates the fluoroscopic image Gnew by the derived rotation angle αnew and displays the fluoroscopic image Gnew on the display 24 (Step ST15), and then the process returns to Step ST11.

In a case in which a determination result in Step ST14 is “No”, the determination unit 35 determines whether or not the sensors 50A to 50D have detected the movement of the C-arm 2 or the movement of the fluoroscopy apparatus 1A (movement detection: Step ST16). In a case in which a determination result in Step ST16 is “No”, the determination unit 35 instructs the display controller 34 to rotate and display the latest fluoroscopic image Gnew by the same rotation angle as the rotation angle in a case in which the previously acquired fluoroscopic image Gold was displayed. Accordingly, the display controller 34 rotates the fluoroscopic image Gnew by the same rotation angle as the rotation angle in a case in which the previously acquired fluoroscopic image Gold was displayed, displays the fluoroscopic image Gnew on the display 24 (Step ST17), and then the process returns to Step ST11.

In a case in which a determination result in Step ST16 is “Yes”, the determination unit 35 instructs the display controller 34 to rotate and display the latest fluoroscopic image Gnew by the latest rotation angle αnew. Accordingly, the display controller 34 rotates the fluoroscopic image Gnew by the rotation angle αnew, displays the fluoroscopic image Gnew on the display 24 (Step ST18), and then the process returns to Step ST11.

In this way, in the second embodiment, in a case in which the C-arm 2 is moved or the fluoroscopy apparatus 1A is moved, the fluoroscopic image Gnew is rotated and displayed by the newly derived rotation angle αnew. Therefore, even in a case in which the rotation angle of the fluoroscopic image G0 is significantly changed due to the movement of the C-arm 2 or the movement of the fluoroscopy apparatus 1A, the fluoroscopic image G0 can be displayed in a desired orientation. Therefore, the visibility of the continuously displayed fluoroscopic images G0 can be improved.

In each of the above embodiments, the radiation image processing device according to the present embodiment comprises the imaging controller 31, but the present disclosure is not limited thereto. The imaging controller 31 may be provided separately from the radiation image processing device according to the present embodiment.

In addition, in the first embodiment, in a case in which the change in the newly derived rotation angle αold from the previously derived rotation angle is less than the reference angle Th0, the fluoroscopic image Gnew is rotated and displayed by the same rotation angle αold as the rotation angle αold in a case in which the previously acquired fluoroscopic image Gold was displayed. However, the present disclosure is not limited thereto. Each time a new fluoroscopic image Gnew is acquired, the new fluoroscopic image Gnew may be rotated and displayed by a newly derived rotation angle αnew.

Moreover, the radiation in each of the embodiments described above is not particularly limited, and a-rays or y-rays can be applied in addition to X-rays.

Further, in above-described embodiment, for example, as hardware structures of processing units that execute various kinds of processing, such as the imaging controller 31, the feature point detection unit 32, the rotation angle derivation unit 33, the display controller 34, and the determination unit 35, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different types of two or more processors (for example, a combination of a plurality of FPGAs or a combination of the CPU and the FPGA). In addition, a plurality of processing units may be configured by one processor.

As an example where a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

The supplementary notes of the present disclosure will be described below.
Supplementary Note 1
A radiation image processing device comprising at least one processor,
in which the processor is configured to:
   acquire a fluoroscopic image captured by a radiation fluoroscopy device having a C-arm;
   detect a plurality of feature points of a structure included in the fluoroscopic image;
      derive a rotation angle of the fluoroscopic image from a reference based on the plurality of detected feature points; and
   rotate and display the fluoroscopic image based on the derived rotation angle.
Supplementary Note 2
The radiation image processing device according to Supplementary Note 1,
in which, in a case in which the radiation fluoroscopy device continuously acquires the fluoroscopic images, the processor is configured to:
   detect the feature points and derive the rotation angle each time the fluoroscopic image is acquired at a predetermined interval; and
   in a case in which a change in a newly derived rotation angle from a previously derived rotation angle is less than a reference angle, rotate and display the fluoroscopic image by the same rotation angle as a rotation angle in a case in which the previously acquired fluoroscopic image was displayed.
Supplementary Note 3
The radiation image processing device according to Supplementary Note 2,
in which the processor is configured to, in a case in which the newly derived rotation angle has changed by the reference angle or more from the previously derived rotation angle, rotate and display the fluoroscopic image by the newly derived rotation angle.
Supplementary Note 4
The radiation image processing device according to Supplementary Note 2 or 3,
in which the processor is configured to:
   detect whether or not the C-arm has moved or the radiation fluoroscopy device has moved; and
   in a case in which the movement of the C-arm or the movement of the radiation fluoroscopy device is detected, rotate and display the fluoroscopic image by the newly derived rotation angle.
Supplementary Note 5
The radiation image processing device according to any one of Supplementary notes 1 to 4,
in which the structure is a plurality of vertebrae,
the feature points are centroids of each of the plurality of vertebrae, and
the processor is configured to:
   derive an approximate straight line connecting the centroids of the plurality of vertebrae; and
   rotate the fluoroscopic image such that the approximate straight line has an orientation of the reference.
Supplementary Note 6
A radiation image processing method executed by a computer, the radiation image processing method comprising:
acquiring a fluoroscopic image captured by a radiation fluoroscopy device having a C-arm;
detecting a plurality of feature points of a structure included in the fluoroscopic image;
deriving a rotation angle of the fluoroscopic image from a reference based on the plurality of detected feature points; and
rotating and displaying the fluoroscopic image based on the derived rotation angle.
Supplementary Note 7
A radiation image processing program causing a computer to execute:
a step of acquiring a fluoroscopic image captured by a radiation fluoroscopy device having a C-arm;
a step of detecting a plurality of feature points of a structure included in the fluoroscopic image;
a step of deriving a rotation angle of the fluoroscopic image from a reference based on the plurality of detected feature points; and
a step of rotating and displaying the fluoroscopic image based on the derived rotation angle.

What is claimed is:

1. A radiation image processing device comprising at least one processor,
wherein the processor is configured to:
acquire a fluoroscopic image captured by a radiation fluoroscopy device having a C-arm;
detect a plurality of feature points of a structure included in the fluoroscopic image;
derive a rotation angle of the fluoroscopic image from a reference based on the plurality of detected feature points; and
rotate and display the fluoroscopic image based on the derived rotation angle.

2. The radiation image processing device according to claim 1,
wherein, in a case in which the radiation fluoroscopy device continuously acquires the fluoroscopic images, the processor is configured to:
detect the feature points and derive the rotation angle each time the fluoroscopic image is acquired at a predetermined interval; and
in a case in which a change in a newly derived rotation angle from a previously derived rotation angle is less than a reference angle, rotate and display the fluoroscopic image by the same rotation angle as a rotation angle in a case in which the previously acquired fluoroscopic image was displayed.

3. The radiation image processing device according to claim 2,
wherein the processor is configured to, in a case in which the newly derived rotation angle has changed by the reference angle or more from the previously derived rotation angle, rotate and display the fluoroscopic image by the newly derived rotation angle.

4. The radiation image processing device according to claim 2,
wherein the processor is configured to:
detect whether or not the C-arm has moved or the radiation fluoroscopy device has moved; and
in a case in which the movement of the C-arm or the movement of the radiation fluoroscopy device is detected, rotate and display the fluoroscopic image by the newly derived rotation angle.

5. The radiation image processing device according to claim 3,
wherein the processor is configured to:
detect whether or not the C-arm has moved or the radiation fluoroscopy device has moved; and
in a case in which the movement of the C-arm or the movement of the radiation fluoroscopy device is detected, rotate and display the fluoroscopic image by the newly derived rotation angle.

6. The radiation image processing device according to claim 1,
wherein the structure is a plurality of vertebrae,
the feature points are centroids of each of the plurality of vertebrae, and
the processor is configured to:
derive an approximate straight line connecting the centroids of the plurality of vertebrae; and
rotate the fluoroscopic image such that the approximate straight line has an orientation of the reference.

7. A radiation image processing method executed by a computer, the radiation image processing method comprising:
acquiring a fluoroscopic image captured by a radiation fluoroscopy device having a C-arm;
detecting a plurality of feature points of a structure included in the fluoroscopic image;
deriving a rotation angle of the fluoroscopic image from a reference based on the plurality of detected feature points; and
rotating and displaying the fluoroscopic image based on the derived rotation angle.

8. A non-transitory computer-readable storage medium that stores a radiation image processing program causing a computer to execute:
a step of acquiring a fluoroscopic image captured by a radiation fluoroscopy device having a C-arm;
a step of detecting a plurality of feature points of a structure included in the fluoroscopic image;
a step of deriving a rotation angle of the fluoroscopic image from a reference based on the plurality of detected feature points; and
a step of rotating and displaying the fluoroscopic image based on the derived rotation angle.
